(19) United States
(12) Patent Application Publication
(10) Pub. No.: US 2015/0157344 A1
(43) Pub. Date: Jun. 11, 2015

(54) MEDICAL RETRIEVAL DEVICES AND RELATED METHODS OF USE

(71) Applicant: Boston Scientific Scimed, Inc., Maple Grove, MN (US)

(72) Inventors: Richard C. TAH, Framingham, MA (US); Kenneth J. DAIGNAULT, Holden, MA (US); Ronald CIULLA, Westford, MA (US)

(73) Assignee: Boston Scientific Scimed, Inc.

(21) Appl. No.: 14/554,686

(22) Filed: Nov. 26, 2014

Related U.S. Application Data
(60) Provisional application No. 61/912,739, filed on Dec. 6, 2013.

(51) Int. Cl. A61B 17/221; A61B 17/50; A61B 18/14

(52) U.S. Cl. CPC A61B 17/221; A61B 18/14; A61B 17/50; A61B 18/1492; A61B 2017/2212; A61B 2018/00595

(57) ABSTRACT

A medical device may include an elongate member including a proximal end, a distal end, and a lumen extending therebetween, wherein a distal end of the lumen includes a wall disposed substantially perpendicular to a longitudinal axis of the elongate sheath, wherein the wall includes at least one opening therethrough. The medical device may also include an actuation assembly operably coupled to the proximal end, an actuating member coupled to a portion of the actuation assembly and extending into the lumen of the elongate member, and at least one loop having a first portion coupled to a distal end of the actuating member and a second portion extending through the at least one opening.

### CROSS-REFERENCE TO RELATED APPLICATIONS

This application claims the benefit of U.S. Provisional Application No. 61/912,739, filed Dec. 6, 2013, the disclosure of which is incorporated herein by reference in its entirety.

### TECHNICAL FIELD

Various embodiments of the present disclosure relate generally to medical devices and related systems and methods. More specifically, the present disclosure relates to devices, systems, and methods for retrieving objects within a patient, including, but not limited, to medical devices and procedures for assisting in the capture and release of tissue or foreign matter from a body portion.

### BACKGROUND

Organic material (e.g., blood clots, tissue, and biological concretions such as urinary, biliary, and pancreatic stones) or inorganic material (e.g., components of medical devices or other foreign matter) may sometimes obstruct or otherwise be present within the body's anatomical lumens, such as, e.g., the biliary tree or a urinary passageway. More specifically, biological concretions can develop in certain parts of the body, such as kidneys, pancreas, and gallbladder. Minimally invasive medical procedures generally involve causing limited trauma to the tissues of the patient, and can be used to remove or otherwise dispose of certain problematic biological concretions or similarly unwanted obstructions.

In addition, lithotripsy and ureteroscopy, for example, also may be used to treat biological concretions (e.g., kidney stones) in the body of patients. Lithotripsy is a medical procedure that uses energy in various forms, such as acoustic shock waves, pneumatic pulsation, electro-hydraulic shock waves, or laser beams, to pulverize biological concretions such as urinary calculi (e.g., kidney stones). The force of the energy, when applied either extracorporeally or intracorporeally, usually in focused and continuous or successive bursts, divides the concretion into smaller fragments that may be extracted from the body, or allowed to pass from the body, for example, through urination.

In certain instances, intracorporeal fragmentation of bodily concretions can become problematic if the stones or stone fragments, instead of progressing out of the patient's body, begin to migrate further into the body or towards an organ. In these circumstances, further medical intervention to prevent aggravation of the patient's condition may become necessary. Therefore, it may be desirable to be able to extract such fragments from the body using a single instrument, preventing the need for successive instrumentation which can cause greater trauma to the patient's tissues.

Many medical retrieval devices can be used to entrap an object, such as a stone fragment, and drag it through an ampulla (i.e., a small dilatation in the patient's anatomical lumen) to remove it. Such medical retrieval devices include, for example, retrieval baskets and may be used through an endoscope, laparoscope, or other suitable introduction sheath. With many known retrieval assemblies, however, it is difficult to release entrapped material, such as a stone, from the retrieval assembly once the material is entrapped. For example, in some patients, a cicatrix or some other constriction that reduces the diameter of a body lumen may form within the lumen. The narrowed lumen may not interfere or prevent normal insertion of a retrieval device. However, after the retrieval device is inserted into the tract, expanded, and an object is captured therein, the diameter of the retrieval assembly containing the entrapped object may exceed the inner diameter of the narrowed lumen. Under these circumstances, it may be desirable to release the entrapped object from within the retrieval assembly so that the retrieval assembly may be safely removed from the lumen. Other circumstances may be encountered during a medical retrieval procedure that may also require releasing an entrapped object from the retrieval device. If the entrapped object cannot be released, more invasive surgical procedures may be needed to disengage the captured object from the retrieval assembly, so that the retrieval assembly may be removed from within the patient.

Further, conventional retrieval assemblies must be advanced beyond or to one side of an object targeted for entrapment, so as to permit entry of the object into the retrieval assembly. Such maneuvers may be technically difficult, especially in narrow anatomical lumens. Further, the walls of a lumen may become attenuated at the side of a lodged object such that advancing a retrieval assembly against the wall may risk rupture of the lumen wall.

The present disclosure provides medical retrieval devices and methods of using the same that address, among others things, the aforementioned shortcomings of the existing devices.

### SUMMARY OF THE DISCLOSURE

The present disclosure includes medical retrieval devices and related methods of use.

In accordance with an embodiment of the disclosure, a medical device may include an elongate member including a proximal end, a distal end, and a lumen extending therebetween, wherein a distal end of the lumen includes a wall disposed substantially perpendicular to a longitudinal axis of the elongate sheath, wherein the wall includes at least one opening therethrough. The medical device may also include an actuation assembly operably coupled to the proximal end, an actuating member coupled to a portion of the actuation assembly and extending into the lumen of the elongate member, and at least one loop having a first portion coupled to a distal end of the actuating member and a second portion extending through the at least one opening.

Various embodiments of the disclosure may include one or more of the following aspects: the at least one opening is disposed along a periphery of the wall; the at least one opening includes two openings in communication with one another; the at least one loop further includes a third portion coupled to the distal end of the actuating member; the second portion is disposed between the first portion and the third portion; the wall is defined by a cap disposed at a distal portion of the lumen of the elongate member; the at least one loop is formed of a shape memory material; the at least one loop includes a plurality of loops defining a retrieval assembly; and the retrieval assembly is configured to transition between a collapsed configuration and an expanded configuration.

In another embodiment, a medical device may include an elongate member including a proximal end, a distal end, and a lumen extending therebetween; an actuation assembly operably coupled to the proximal end of the elongate member; an actuating member coupled to a portion of the actuation assembly and extending into the lumen of the elongate member; a cap secured at a distal portion of the lumen of the elongate member, wherein the cap includes a distal end face including a plurality of openings disposed about a periphery of the distal end face; and a plurality of loops, wherein each of the loops includes: a first portion coupled to a distal end of the actuating member; a second portion extending from the first portion and through at least one of the plurality of openings; and a third portion extending from the second portion, through another one of the plurality of openings, and coupled to the distal end of the actuating member.

Various embodiments of the medical device may include one or more of the following features: the plurality of loops define a retrieval assembly configured to transition between a collapsed configuration and an expanded configuration; at least one of the plurality of loops is formed of a super elastic material or a shape memory material; the plurality of openings are disposed in the distal end face in three sets of two; the openings of a single set of openings are in communication with one another; at least one of the openings of the plurality of openings is centered about a longitudinal axis that is at an angle relative to a longitudinal axis of the elongate sheath; and at least one of the plurality of loops is configured to cauterize tissue.

In another embodiment, a method for manipulating an object with a patient's body may include positioning a distal end of a medical device adjacent the object may include an elongate member including a proximal end, a distal end, and a lumen extending therebetween, wherein a distal end of the lumen includes a wall disposed substantially perpendicular to a longitudinal axis of the elongate sheath, wherein the wall includes a plurality of openings therethrough; an actuation assembly operably coupled to the proximal end; an actuating member coupled to a portion of the actuation assembly and extending into the lumen of the elongate member; and a plurality of loops, wherein each of the loops includes: a first portion coupled to a distal end of the actuating member; a second portion extending from the first portion and through at least one of the plurality of openings; and a third portion extending from the second portion, through another one of the plurality of openings, and coupled the distal end of the actuating member. The method may further include moving the actuating member relative to the elongate sheath to transition the plurality of loops from a first configuration to a second configuration and positioning the object within the plurality of loops.

Various embodiments of the method may include one or more of the following features: the first configuration is collapsed configuration and the second configuration is an expanded configuration; further comprising moving the actuating member relative to the elongate sheath to transition the plurality of loops from the second configuration to the first configuration; and the wall is defined by a cap received in a distal portion of the lumen of the elongate sheath.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is a schematic illustration of an exemplary medical device in accordance with an embodiment of the present disclosure.

FIG. 2 is a perspective view of a distal portion of the exemplary medical device of FIG. 1 in a first configuration.

FIG. 3 is a perspective view of the distal portion of the exemplary medical device of FIG. 1 in a second configuration.

FIG. 4 is a perspective of the distal portion of the exemplary medical device of FIG. 1 having an object entrapped therein.

FIG. 5A depicts a top view of a cap for use with the distal portion of the exemplary medical device of FIG. 1.

FIG. 5B depicts a side view of the cap of FIG. 5A.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts.

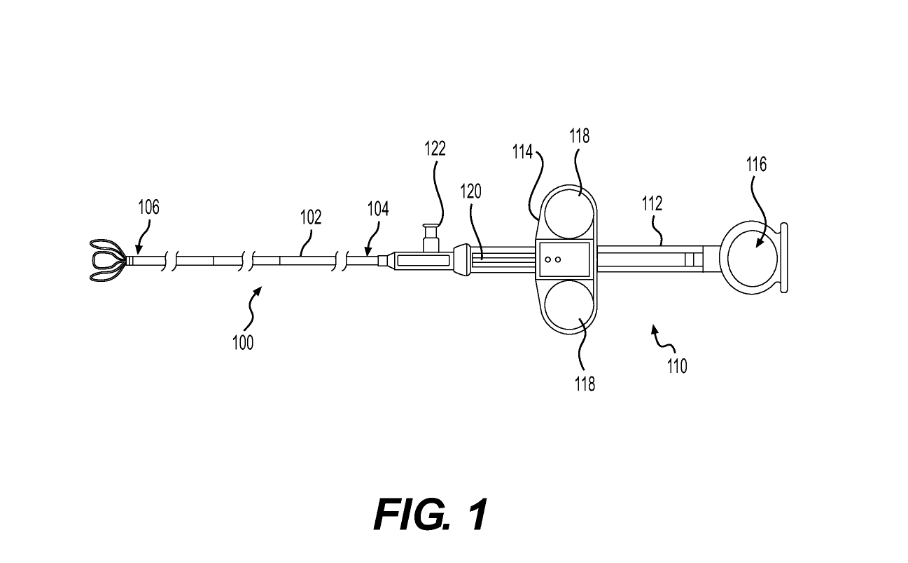

With reference to FIG. 1, there is depicted an exemplary embodiment of a medical device 100, in accordance with the principles of the present disclosure. Medical device 100 may include an elongate sheath 102 having a proximal end 104, a distal end 106, and a lumen 108 extending therebetween.

Elongate sheath 102 may include any suitable configuration desired. For example, elongate sheath 102 may include a generally circular cross-sectional configuration. However, elongate sheath 102 may include any suitable cross-sectional configuration, including, but not limited to, rectangular, square, or triangular. Regardless of the cross-sectional configuration, elongate sheath 102 may include a substantially atraumatic outer configuration, so as to minimize trauma to anatomical structures when elongate sheath 102 is passed into and withdrawn from a patient's body. Lumen 108 may be generally circular in configuration. In some embodiments, however, lumen 108 may correspond to the cross-sectional geometry of elongate sheath 102. Although the depicted embodiment illustrates a single lumen 108 extending through elongate sheath 102, elongate sheath 102 may include any suitable number of lumens therein. In embodiments with a plurality of lumens (not shown) disposed are within elongate sheath 102, the lumens may be completely separated from one another along an entire length of elongate sheath 102, or may be in fluid communication along one or more portions of elongate sheath 102. For example, a distal portion of each of a plurality of lumens extending through sheath 102 may be in fluid communication with one or more adjacent lumens.

Further, elongate sheath 102 may be flexible along its length. In one embodiment, a first portion of elongate sheath 102 may be greater or less flexible than a second portion of elongate sheath 102. For example, a proximal portion 104 of elongate sheath 102 may be rigid as compared to a distal portion 106 of elongate sheath 102. In a further embodiment, a central portion of elongate sheath 102 may exhibit a differing flexibility as compared to one or both of proximal and distal portions 104, 106.

One or more portions of elongate sheath 102 may be transparent, translucent, and/or opaque to light. For example, a distal portion of elongate sheath 102 adjacent distal end 102 may be transparent. Those of ordinary skill will readily understand that similar principles may also apply to radiopacity. For example, a distal portion of elongate sheath 102 may be radiotransparent or otherwise radiolucent so as to allow an operator to visualize structure within elongate sheath 102 with suitable imaging devices. Elongate sheath 102 may include one or more radiopaque markers (not shown) disposed on an outer surface thereof. The radiopaque markers may be any suitable markers known in the art, including, but not limited to, marker bands made of thin-walled metal sheets or tubes placed at strategic locations along an outer surface of elongate sheath 102.

One or more surfaces of elongate sheath 102 may include any suitable coating or covering known in the art. For example, an outer surface of elongate sheath 102 may include a suitable therapeutic coating, such as, e.g., an anesthetic or hemostatic agent. Similarly, an inner surface of elongate sheath 102 may include a suitable lubricious coating. Further, a portion (e.g., a portion adjacent distal end 106) of elongate sheath 102 may include one or more anchoring mechanisms configured to center or otherwise stabilize elongate sheath 102 within an anatomical lumen. For example, a distal portion of elongate sheath 102 may include a plurality of expandable members (not shown) disposed along a length thereof for selectively engaging a wall of an anatomical lumen. The expandable members may be inflatable balloons, expandable mechanical assemblies (e.g., cages or foams), or any suitable mechanism known in the art. In some embodiments, a wall of elongate sheath 102 may define lumen 108. The wall may be reinforced by any suitable means. For example, the wall of sheath 102 may includes a plurality of structures disposed therein to provide, among other things, rigidity and/or reinforcement. In some embodiments, the structures may includes, but are not limited to, braided filaments comprising metal or polymer.

In some embodiments, a distal end portion of elongate sheath 102 may be selectively steerable. In such embodiments, medical device 100 may include suitable articulating mechanisms for effecting desired positioning and/or steering of elongate sheath 102. For example, elongate sheath 102 may include a plurality of steering wires (not shown) extending to and coupled to, e.g., distal end 106, such that pulling proximally on one or more of the steering wires causes the distal end 106 to accordingly bend. In such embodiments, elongate sheath 102 may exhibit flexibility to permit selective steering but sufficient rigidity to allow insertion of elongate sheath 102 into a patient's anatomical lumens. Still further, elongate sheath 102 may be made of a plurality of articulating components (e.g., joints defining a lumen therethrough) movably coupled to one another.

A proximal end 104 of elongate sheath 102 may be operably coupled to an actuation assembly 110. In some embodiments, actuation assembly 110 may be detachably coupled to elongate sheath 102. Actuation assembly 110 may be any suitable actuation assembly known in the art. For example, actuation assembly 110 may be configured for one-handed operation.

With continuing reference to FIG. 1, actuation assembly 110 may define a base 112 and an actuation mechanism 114 movably coupled to base 112 via any suitable mechanism. A proximal end portion of base 112 may define a suitable opening 116 for receiving, e.g., a thumb, of an operator. Actuation mechanism 114, on the other hand, may define a plurality of openings 118 for receiving, e.g., an index and middle fingers, of the operator. As alluded to above, actuation mechanism 114 may be configured for reciprocal movement relative to base 112. Further, actuation mechanism 114 may be operably coupled to an actuating member 120 via any suitable mechanism. In some embodiments, actuating member 120 may be detachably attached to, e.g., a distal portion of actuation mechanism 114 via any suitable means known in the art.

Actuating member 120 may include any suitable actuating member known in the art. For example, actuating member 120 may include a single pull-wire or a plurality of wires braided together to form actuating member 120. Further, actuating member 120 may include a rigid rod configured for reciprocal movement within elongate sheath 102.

Actuating member 120 may extend from a distal portion of actuation mechanism 114 to a distal portion of elongate sheath 102. In some embodiments, an outer surface of actuating member 120 may include a suitable coating, such as, e.g., a lubricious coating, to facilitate movement of actuating member 120 within elongate sheath 102. In some embodiments, actuating member 120 may be configured to conduct energy, such as, e.g., electrical or radiofrequency (RF) energy. In such embodiments, one or more portions of actuating member 120 may include an insulating covering or coating. Further, in embodiments where actuating member 120 is electrically conductive, a portion of actuating member 120 may be electrically coupled to a connector 122 on base 112. Connector 122 may be any suitable mechanism (e.g., an electrical connector or an RF connector) known in the art for receiving a plug of a desired energy generator (not shown).

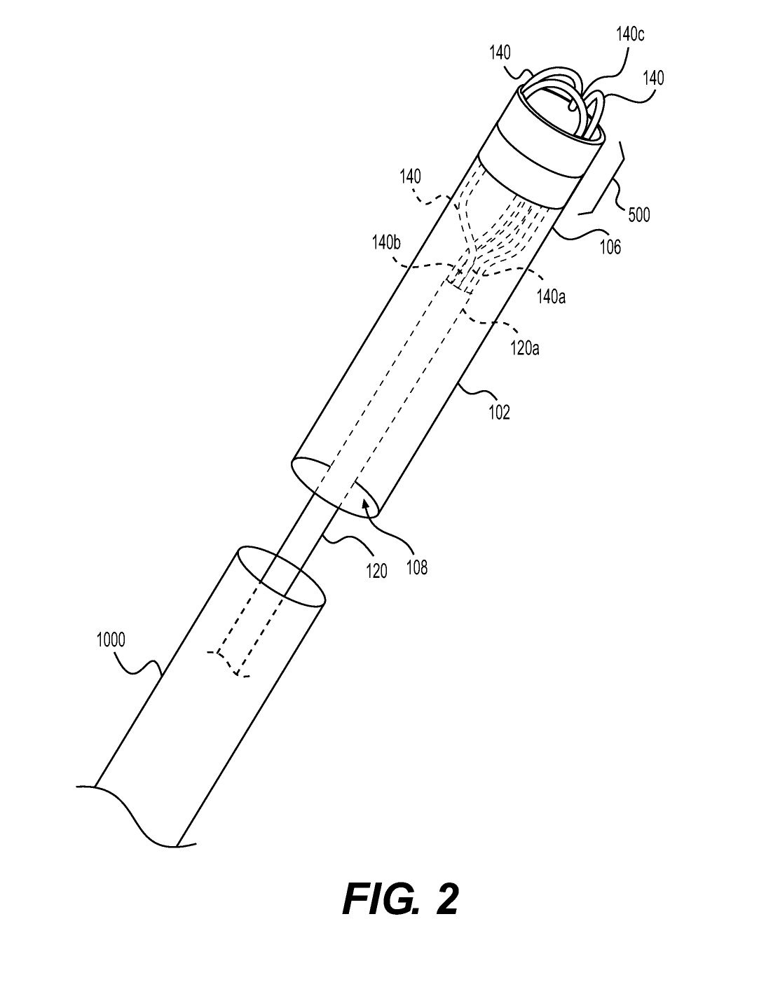

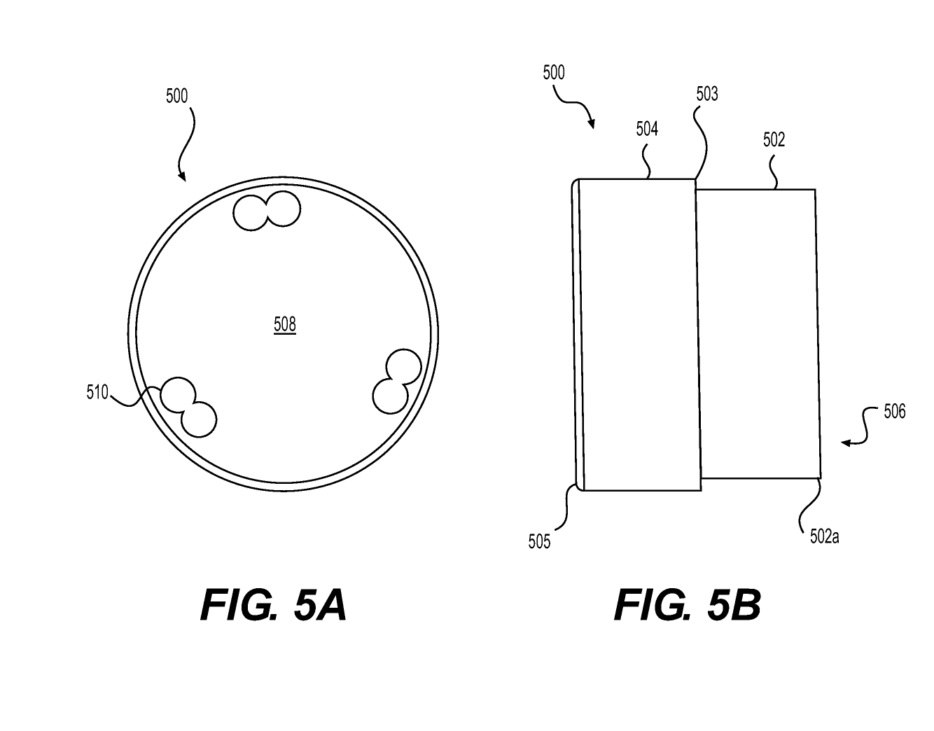

With reference now to FIG. 2, a distal end 106 of elongate sheath 102 may be coupled to a cap 500 (shown in FIGS. 5A and 5B). Cap 500 may include any suitable configuration known in the art. In one embodiment, cap 500 may include a first portion 502 and a second portion 504. First portion 502 may be disposed proximally of second portion 504. A step 503, which is formed by a radially outward extension of second portion 504, may be disposed between first portion 502 and second portion 504.

First portion 502 may include any suitable configuration. For example, first portion 502 may define a substantially circular cross-sectional configuration. First portion 502 also may include any suitable dimension. For example, first portion 502 may define a diameter configured to be received within lumen 108 of elongate sheath 102. That is, a diameter of first portion 502 may be smaller (albeit, slightly) than a diameter of lumen 108. Further, first portion 502 may be configured to be frictionally retained within lumen 108. For example, first portion 502 may define a geometry that corresponds to an inner geometry of lumen 108. A proximal end 502a of first portion 502 may define an opening 506 leading to a channel defined by an interior of cap 500. In an alternative embodiment, distal end 106 of elongate sheath 102 may be received within opening 506 to secure cap 500 on elongate sheath 102. In such embodiments, opening 506 may include a diameter slightly larger than an outer diameter of elongate sheath 102. Cap 500 may be secured to elongate sheath 102 via any suitable means known in the art. In some embodiments, cap 500 may be secured via a friction or interference fit. In other embodiments, cap 500 may be glued to elongate sheath 102. Still further, cap 500 may be welded to elongate sheath 102. Moreover, cap 500 and elongate sheath 102 may be made of a one-piece configuration.

Second portion 504 also may include any suitable configuration. For example, second portion 504 may define a substantially circular cross-sectional configuration. Second portion 504 also may include any suitable dimension. For example, second portion 504 may include a diameter that is larger than the diameter of first portion 502. The difference in diameters of first portion 502 and second portion 504 may correspond to a thickness of elongate sheath 102, such that when first portion 502 is received within lumen 108, an outer surface of second portion 504 is flush with an outer surface of elongate sheath 102, as shown in, e.g., FIG. 2. A distal end of second portion 504 may include a suitable atraumatic configuration. For example, as shown in FIG. 5B, a distal end of second portion 504 may include a chamfered or otherwise beveled edge 505 extending about a portion or an entire periphery of second portion 504.

A distal end of cap 500 may include a wall defined by distal end face 508. Distal end face 508 may be substantially planar and may close off the distal end of cap 500 so that channel 506 is not a through channel. In one embodiment, distal end face 508 may be substantially flush with a distal end of cap 500. In other embodiments, however, distal end face 508 may be recessed into second portion 504. The wall defining distal end face 508 may be disposed perpendicular to or substantially perpendicular (e.g., at an angle of approximately 90 degrees) to a central longitudinal axis of cap 500. In further embodiments, distal end face 508 may be at any suitable angle relative to the central longitudinal axis of cap 500.

Further, in some embodiments, distal end face 508 may include a plurality of openings 510. Openings 510 may include any suitable configuration and/or dimension. In one embodiment, for example, openings 510 may include a substantially circular configuration. In another embodiment, each of openings 510 may include two substantial semi-circular portions in communication with one another. Further, one or more opening 510 of the plurality of openings 510 may have a differing configuration than the remaining openings 510. Alternatively, each of the plurality of openings 510 may have a substantially similar configuration. As shown in FIG. 5A, one or more openings 510 may be in communication with each other. For example, two openings 510 may be in communication. In some embodiments, openings 510 may be replaced with a single opening extending around a periphery of distal end face 508.

Openings 510 may be disposed on distal end face 508 in any suitable configuration. In one embodiment, as shown in FIG. 5A, openings 510 may be arranged in pairs of two disposed equally around a periphery of distal end face 508. The spacing between adjacent openings 510 may be varied as desired. Distal end face 508 may include an additional opening (not shown) in the center of distal end face 508. The center opening may include a dislodging member (not shown) for forcing captured objects out of the retrieval assembly 142 discussed herein. Further, openings 510 may be disposed about axes that are parallel to a central longitudinal axis (not shown) of cap 500. Alternatively, openings 510 may extend along an axis (not shown) that is at an angle to the central longitudinal axis of cap 500. In such embodiments, openings 510 may include a tapering configuration configured to urge loops 140 (discussed below in greater detail) in a predetermined direction or orientation. Openings 510 may be positioned and configured to reduce the risk of adjacent loops 140 becoming entangled with one another during operation. As such, openings 510 may be configured to maintain equal spacing between loops 140 or portions thereof such that substantially similar gaps are maintained between adjacent wires of loops 140. Openings 510 may also act as pivot points for loops 140 while loops 140 are being retracted into sheath 102 so as to make loops 140 bend towards the central longitudinal axis of cap 500, so as to grip an entrapped object.

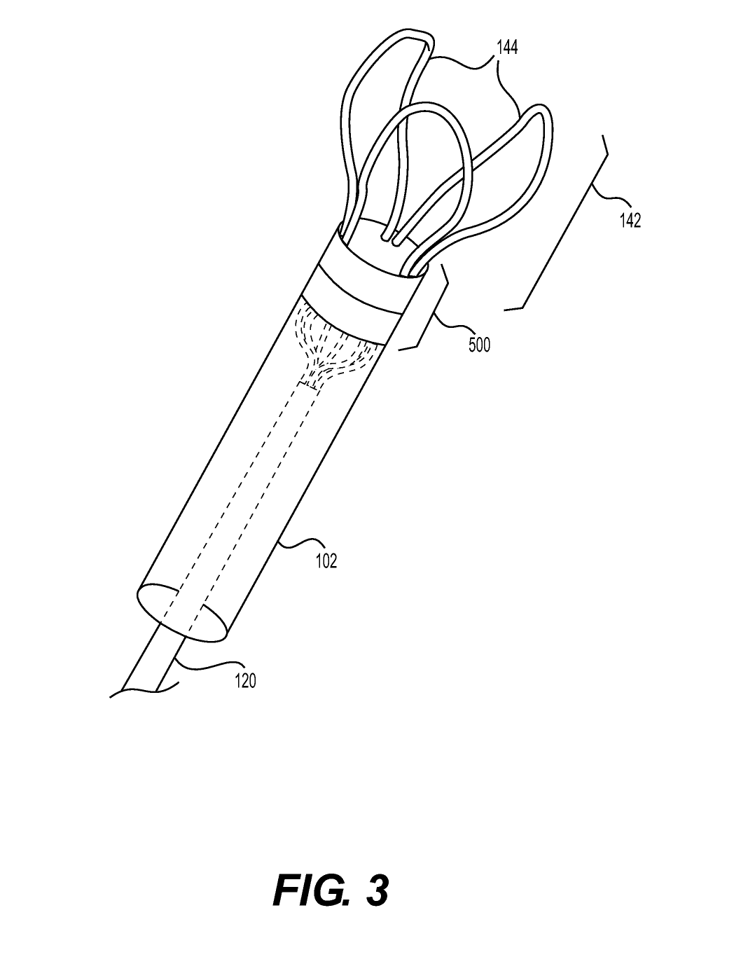

With renewed reference to FIG. 2, a distal end 120a of actuating member 120 may be operably coupled to a plurality of flexible loops 140. Although the depicted embodiment illustrates three loops 140, a larger or smaller number of loops 140 may be provided. Together, loops 140 define a distal retrieval assembly 142 (FIG. 3). Loops 140 may be made of any suitable flexible material. In one embodiment, loops 140 may be made of a single wire of stainless steel, for example. In another embodiment, loops 140 may be made of a shape memory or superelastic material, including, but not limited to, nitinol. In some embodiments, loops 140 may be made of a material configured to withstand the energies used in lithotripsy procedures. Further, loops 140 may be made of a plurality of wires 140 braided together. Further, loops 140 may include any suitable coating and/or covering known in the art. For example, a portion of one or more loops 140 may include a coating configured to increase friction on an outer surface thereof. Moreover, each of loops 140 may include one or more radiopaque portions to facilitate visualization when inserted into a patient. One or more of loops 140 may be made of any suitable filament or wire. In addition, loops 140 may include any suitable cross-sectional shape and/or configuration, including, but not limited to, circular, flat, rectangular, square, and/or triangular. Further, a shape, configuration, and/or dimension of one or more of loops 140 may be varied along the length of the one or more of loops 140 so as to, e.g., impart particular mechanical properties. More particularly, a first portion of a loop 140 may have a first cross-sectional shape (or dimension) and a second portion of a loop 140 may have a second cross-section shape (or dimension). Such a configuration may results in a loop 140 having varying degrees of flexibility or stiffness along a length of loop 140. For example, a proximal portion of loop 140 may be relatively stiff as compared to a distal portion of loop 140, or vice versa.

Each of loops 140 may include a first portion 140a, a second portion 140b, and a third portion 140c connecting the first and second portions 140a, 104b together. When assembled, first portion 140a may extend distally away from distal end 120a of actuating member 120, third portion 140c may “loop” through a first opening 510 and back into a second opening 510, and a second portion 140b may extend proximally toward distal end 120a. That is, a portion of third portion 140c may be disposed on a first side of distal end face 508 while portions of first and second portions 140a, 140b are disposed on a second side of distal end face 508. First and second portions 140a, 140b may be coupled to distal end 120a via any suitable means known in the art. For example, in one embodiment, first and second portions 140a, 140b may be welded or otherwise mechanically coupled to distal end 120a. In a further embodiment, loops 140 and actuating member 120 may be made of a one-piece construction. For example, proximal extensions of both first and second portions 140a, 140b of each of loops 140 may be braided together to form actuating member 120, which, at its proximal end, may be coupled to base 112 as discussed above.

A portion (e.g., third portion 140c) of one or more loops 140 may include geometric configurations suitable for enhancing an ability of retrieval assembly 142 to grasp, maintain, or otherwise retain objects and tissue within retrieval assembly 142. For example, third portion 140c may include a plurality of hooks, barbs, or spikes (not shown) configured to facilitate grasping of an object or tissue. Third portion 140c may also include a sharpened outer edge (not shown) configured to assist with scraping or prying objects embedded within the walls of an anatomical lumen. One or more loops 140 may have any suitable extension (e.g., a needle tip) attached to a distal portion.

In some embodiments, inner edges 144 of loops 140 may be sharpened such that as loops 140 are closed about tissue, edges 144 cut the tissue into smaller pieces. In such embodiments, retrieval assembly 142 may function as a morcellator. Further, in another embodiment, loops 140 may be configured to cauterize tissue upon the application of energy to connector 122. In such an embodiment, loops 140 may also cut through tissue as they close about the tissue captured within retrieval assembly 142.

With reference to FIGS. 1-4, an exemplary assembly of the aforementioned components will be now described. Elongate sheath 102 may extend distally from actuation assembly 110. A proximal end of actuation element 120 may be coupled to actuation mechanism 114, which may be movably mounted to base 112. A distal end 120a of actuation member 120 may extend through elongate sheath 102 and operably couple to portions 140a, 140b of a plurality of loops 140. As shown in FIGS. 2-4, each of loops 140 may include a third portion 140c that extends through on opening 510 in distal end face 508 of cap 500 and back through another opening 510. As discussed above, cap 500 may be secured to a distal end 106 of elongate sheath 102.

In use, advancing actuation mechanism 114 distally relative to base 112 may cause actuation member 120 to move distally relative to elongate sheath 102, which pushes each third portion 140c of loop 140 distally out of and away from cap 500. In embodiments where loops 140 are made of super elastic or shape memory materials, loops 140 may assume a predetermined configuration, such as, e.g., the configuration depicted in FIG. 3. In one embodiment, e.g., the predetermined configuration may include each of loops 140 bending radially outward and away from a central longitudinal axis of elongate sheath 102. Further, selective orientation and/or positioning of openings 510 may assist in positioning each of loops 140 as they move distally away from cap 500.

In an alternative assembly, actuating member 120 may be coupled to base 112 and elongate sheath 120 may be coupled to actuation mechanism 114. In such an embodiment, therefore, moving actuation mechanism 114 relative to base 112 will cause elongate sheath 102 to move relative to actuating member 120.

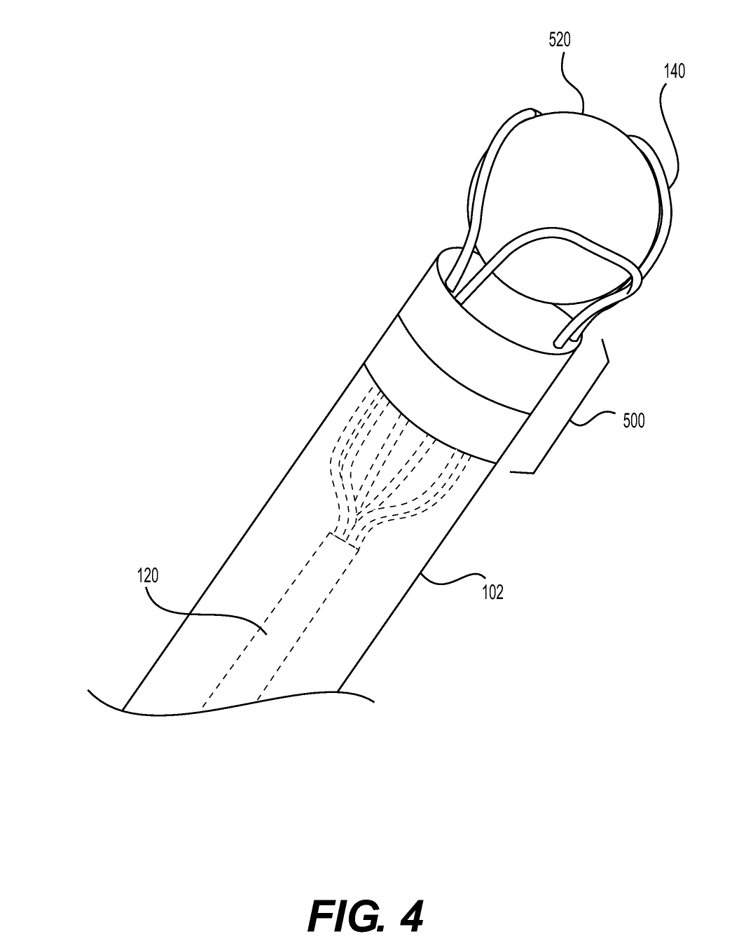

In some embodiments, the disclosed medical device 100 may include at least one locking feature (not shown) operably coupled with either actuation assembly 110 or cap 500. The locking feature may be configured to secure loops 140 in a deployed position once extended out of cap 500, as shown in FIG. 3. For example, in use, once an object 520 (shown in FIG. 4) is entrapped between loops 140 of retrieval assembly 142, the locking feature may be actuated to hold loops 140 in their extended position, thereby ensuring object 520 is securely retained within retrieval assembly 142. If it becomes necessary to release object 520, the locking feature may be deactivated so loops 140 may be retracted within cap 500.

In another embodiment, cap 500 may be rotated relative to elongate sheath 102 and a proximal portion of loops 140 prior to use. As a result, when actuated, loops 140 may exit cap 500 in a twisted, and may impart varied forces upon targeted objects. For example, in embodiments where cap 500 is rotated relative to sheath 102 prior to use, loops 140 may exit cap 500 in a twisted configuration and may therefore impart a stronger force on an entrapped object 520.

In a further embodiment, medical device 100 may include a plurality of interchangeable caps 500. Each of the plurality of caps 500 may include openings (as described above) through which loops 140 may extend. However, it is contemplated that the configuration, disposition, number, shape, and/or size of the openings on each of the plurality of caps 500 may be different so as to produce differing configurations of retrieval assembly 142 when loops 140 are deployed through each cap. Thus, a user of medical device 100 may be able to selectively control a deployment configuration/pattern of loops 140 by selecting one of the plurality of caps 500. For example, with loops 140 withdrawn into elongate sheath 102, a user may selectively place a desired cap 500 known to correspond to a particular configuration on the distal end of sheath 102, align the loops 140 with the openings, and then extend the loops 140 through the openings. In one example, the openings may cause the loops 140 to radially flare away from one another when they are extended through cap 500.

In use, actuating mechanism 114 may be moved proximally relative to base 112, which causes elongate sheath 102 to move proximally relative to actuating member 120 and each of loops 140, thereby retracting elongate sheath 102 relative to retrieval assembly 142. During any of the alternative assemblies and actuation methods, openings 510 may be configured to position loops 140 apart and away from the longitudinal axis of cap 500, thereby opening retrieval assembly 142.

The disclosed medical devices may be utilized in any suitable application requiring the capture and removal of materials from the body. The disclosed medical devices may be simple and inexpensive to manufacture, and have improved durability as they may have fewer components prone to failure.

Any aspect set forth in any embodiment may be used with any other embodiment set forth herein. The devices and apparatus set forth herein may be used in any suitable medical procedure, may be advanced through any suitable body lumen and body cavity, and may be used to remove material from any suitable body portion. For example, the apparatuses and methods described herein may be used through any natural body lumen or tract, including those accessed orally, vaginally, rectally, nasally, urethrally, or through incisions in any suitable tissue.

In one embodiment, medical device 100 may be inserted into a patient's anatomical lumen, such as, e.g., a biliary passage. Medical device 100 may be inserted into the patient with the aid of any suitable introduction sheath 1000 (shown in FIG. 2), which may include, but is not limited to, an endoscope, laparoscope, ureteroscope, and/or bronchoscope. Once a distal portion of medical device 100 is positioned adjacent an object 520 targeted for retrieval, an operator may move actuation mechanism 114 distally relative to base 112 to advance loops 140 out of and away from cap 500 to “open” retrieval assembly 142, as discussed above. The operator may then manipulate medical device 100 to position object 520 in between of the spread-apart loops 140. Subsequently, the operator may retract actuation mechanism 114 proximally relative to base 112 to pull actuation member 120 proximally relative to elongate sheath 102 and withdraw each of loops 140 into elongate sheath 102 through cap 500. Doing so will cause loops 140 to collapse about object 520, thereby entrapping object 520 within retrieval assembly 142. Medical device 100 along with entrapped object 520 may be then removed from the patient. Alternatively, object 520 may be fragmented during, e.g., a lithotripsy procedure. Should it become necessary to release object 520 from within retrieval assembly 142, actuation mechanism 114 may be move distally relative to base 112 to once again spread-apart loops 140 and open retrieval assembly 142.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only. The following disclosure identifies some other exemplary embodiments.

We claim:

1. A medical device, comprising:
an elongate member including a proximal end, a distal end, and a lumen extending therebetween, wherein a distal end of the lumen includes a wall disposed substantially perpendicular to a longitudinal axis of the elongate sheath, wherein the wall includes at least one opening therethrough;
an actuation assembly operably coupled to the proximal end;
an actuating member coupled to a portion of the actuation assembly and extending into the lumen of the elongate member; and
at least one loop having a first portion coupled to a distal end of the actuating member and a second portion extending through the at least one opening.

2. The medical device of claim 1, wherein the at least one opening is disposed along a periphery of the wall.

3. The medical device of claim 1, wherein the at least one opening includes two openings in communication with one another.

4. The medical device of claim 1, wherein the at least one loop further includes a third portion coupled to the distal end of the actuating member.

5. The medical device of claim 4, wherein the second portion is disposed between the first portion and the third portion.

6. The medical device of claim 1, wherein the wall is defined by a cap disposed at a distal portion of the lumen of the elongate member.

7. The medical device of claim 1, wherein the at least one loop is formed of a super elastic material.

8. The medical device of claim 1, wherein the at least one loop includes a plurality of loops defining a retrieval assembly.

9. The medical device of claim 1, wherein the retrieval assembly is configured to transition between a collapsed configuration and an expanded configuration.

10. A medical device, comprising:
an elongate member including a proximal end, a distal end, and a lumen extending therebetween;
an actuation assembly operably coupled to the proximal end of the elongate member;
an actuating member coupled to a portion of the actuation assembly and extending into the lumen of the elongate member;
a cap secured at a distal portion of the lumen of the elongate member, wherein the cap includes a distal end face including a plurality of openings disposed about a periphery of the distal end face; and
a plurality of loops, wherein each of the loops includes:
a first portion coupled to a distal end of the actuating member;
a second portion extending from the first portion and through at least one of the plurality of openings; and
a third portion extending from the second portion, through another one of the plurality of openings, and coupled to the distal end of the actuating member.

11. The medical device of claim 10, wherein the plurality of loops define a retrieval assembly configured to transition between a collapsed configuration and an expanded configuration.

12. The medical device of claim 10, wherein at least one of the plurality of loops is formed of a super elastic material.

13. The medical device of claim 10, wherein the plurality of openings are disposed in the distal end face in three sets of two.

14. The medical device of claim 13, wherein the openings of a single set of openings are in communication with one another.

15. The medical device of claim 10, wherein at least one of the openings of the plurality of openings is centered about a longitudinal axis that is at an angle relative to a longitudinal axis of the elongate sheath.

16. The medical device of claim 10, wherein at least one of the plurality of loops is configured to cauterize tissue.

17. A method for manipulating an object with a patient's body, the method comprising:
positioning a distal end of a medical device adjacent the object, the medical device comprising:
an elongate member including a proximal end, a distal end, and a lumen extending therebetween, wherein a distal end of the lumen includes a wall disposed substantially perpendicular to a longitudinal axis of the elongate sheath, wherein the wall includes a plurality of openings therethrough;
an actuation assembly operably coupled to the proximal end;
an actuating member coupled to a portion of the actuation assembly and extending into the lumen of the elongate member; and
a plurality of loops, wherein each of the loops includes:
a first portion coupled to a distal end of the actuating member;
a second portion extending from the first portion and through at least one of the plurality of openings; and
a third portion extending from the second portion, through another one of the plurality of openings, and coupled the distal end of the actuating member;
moving the actuating member relative to the elongate sheath to transition the plurality of loops from a first configuration to a second configuration; and
positioning the object within the plurality of loops.

18. The method of claim 17, wherein the first configuration is collapsed configuration and the second configuration is an expanded configuration.

19. The method of claim 17, further comprising moving the actuating member relative to the elongate sheath to transition the plurality of loops from the second configuration to the first configuration.

20. The method of claim 17, wherein the wall is defined by a cap received in a distal portion of the lumen of the elongate sheath.
